(19) United States
(12) Patent Application Publication
(10) Pub. No.: US 2023/0241457 A1
(43) Pub. Date: Aug. 3, 2023

(54) WEARABLE ELASTIC BIO-SENSORS FOR IMPROVED EMERGENCY CARE

(71) Applicant: Richard Postrel, Miami Beach, FL (US)

(72) Inventor: Richard Postrel, Miami Beach, FL (US)

(21) Appl. No.: 18/102,763

(22) Filed: Jan. 29, 2023

Related U.S. Application Data
(60) Provisional application No. 63/304,610, filed on Jan. 29, 2022.

(51) Int. Cl. A63B 24/00

(52) U.S. Cl. CPC A63B 24/0087; A63B 24/0075; G16H 20/30

(57) ABSTRACT

The present invention provides methods and tools for improving training regimes and optimizing exercise effects. It is ideally suited for rehabilitation services, sports medicine, gym workouts, occupational therapy, physical therapy, and training setups including especially arduous endeavors or occupations, such as military personnel, fighter pilots, astronauts, law enforcement, intense athletes, etc. This invention provides enhanced biofeedback towards a complete and comprehensive understanding of the body's real-time metabolic status and capabilities. Specialists, such as cardiologists and vascular surgeons, will find the simultaneous feedback from separate quadrants of the body especially useful in predicting future events. A series of wearable wireless nanosensor appliances on the body and/or in handlebars, grips, gloves, etc., monitors biomolecules emitted from hands, ankles, fingers, head, torso, etc. The analytical system assesses real-time metabolic status of the individual. Both small and large molecules can be analyzed, e.g., to indicate the fuels being used (e.g., proteins, sugars, fats), tissue breakdown products (including, but not limited to skeletal muscle, cardiac muscle, ketones, fat cells, etc.), hormones, liver and lung metabolites, etc. Devices of the invention use highly sensitive compound selective nanosensing elements to produce real-time status and summary reports immediately available to the practicing individual, coach, manager, teammate, therapist, and/or other selected party of interest.

The present invention greatly improves emergency care in crisis situations. Patients experiencing heart attacks, life-threatening accidents, or conflict related battlefield injuries where time to diagnosis and treatment are critical to survival will greatly benefit from this invention. The invention features sets of elastic bands of different sizes that are embedded with various biosensors for easy and rapid deployment on the patient's wrists, ankles, head and torso. One of the major attributes of this invention is that it can be fully deployed and operational in less than one minute. This system and method eliminates the need for placing numerous electrodes on a patient which are frequently dislodged, require skilled personnel, and can take up to 10 minutes to deploy. Another key attribute of this invention is richer quantities of data from all sections of the body simultaneously in real-time. Data is streamed through the patient's skin and aggregated at a central station on the patient's chest or torso for further transmission to a forward base, hospital, specialist, or trauma center. The platform allows for interactive communication with those third parties for instructions, queries, and recommendations.

The present invention provides exceptional benefit for training and rehabilitation regimens to optimize performance for non-critical patient outcomes. This solution allows for continuous monitoring without the inconvenience of wires and electrodes attached to the patient's body enabling freedom of movement and comfortable sleep while being monitored. Rehabilitation services, sports medicine, gym workouts, occupational therapy, physical therapy, and training setups for exceptionally arduous endeavors or occupations, such as military personnel, fighter pilots, astronauts, law enforcement, etc., benefit from real-time interactive input. Battlefield medics and first responders, such as ambulatory care givers, EMTs, fire fighters, and the like, will dramatically improve patient outcomes and save lives through the immediacy and richness of data provided by this invention. Because patient data is immediately available and communicated to first responders, hospital staff, battlefield command center, etc., this invention allows remote specialist(s) to provide real-time feedback to emergency care giver(s) on site thereby immediately improving the quality of care and patient survivability. This invention features electronically enhanced expandable bands for rapid deployment on patients' wrist, ankles, head, and torso, eliminating the need for applying multiple electrodes (chest only) that deliver far less information and require far more time and skill to deploy.

Each bio-band is embedded with multiple biosensors of various select types that include, but not be limited to: carbon nano-tubules, graphene based sensing devices, pO₂ sensor, motion sensors, pulse, respiration, etc. The integrated system allows for coordinated data acquisition and real-time bio-status by using the patient's skin for transmitting data from each of the applied bio-bands to a central station embedded in the chest or torso band applied to the patient. The central station transmits patient's information from each of the applied bio-bands in real-time to both the care giver(s) on site as well as remote hospitals, specialists, or command centers awaiting the arrival of the patient in critical condition. The data can be enhanced with artificial intelligence and machine learning to improve or suggest optimal treatment(s).

This enhanced biofeedback solution is also well-suited for non-emergency events including, but not limited to: rehabilitation services, sports medicine/training, gym workouts, occupational therapy, physical therapy, in home personal training, etc. By incorporating virtual reality and enhanced reality applications, this invention may include additional devices, such as headgear, sensor enhanced handlebar grips, gloves, and the like. Cameras, in concert with the integrated bio-feedback system, allow for the use of artificial intelligence and machine learning to monitor individuals or groups to enhance participation, learning, and physical performance. Systems and devices of this invention can work in concert with commercial monitoring programs including, but not limited to: Peloton®, Nordic Track®, Echelon Connect®, My Fitness Myx™, Mirror™, Proform Studio® Bike, etc.

This integrated bio-feedback system analyzes the biomolecules produced during training or therapy to assess the real-time metabolic status of the individual. Sensing elements include both single wall carbon nanotubes (SWNTs) and graphene sheets to analyze both small and large molecules. The sensor outputs indicate factors including, but not limited to: the fuels being used (e.g., proteins, sugars, fats), tissue breakdown products (e.g., from skeletal muscle, cardiac muscle, fat cells, etc.), blood sugar, hormones, renal function, water stress, liver and lung metabolites, etc. The devices of the invention use highly sensitive compound selective nanosensing elements to produce real-time status and summary reports immediately available to the practicing individual, coach, manager, team mate, therapist, and/or other selected party of interest. Real-time reporting can be transmitted to the individual using audio, video, tactile or other interfaces.

Wounded warriors, accident victims as well as amateur and professional athletes, aiming to optimize their recovery and performance will benefit from this invention. Occupational therapy, physical therapy and rehabilitation services can be enhanced by maximizing regimens for prescribed activities and/or by greatly facilitating instruction coming from a therapist remote from the patient. Individuals training for specific goals such as a marathon or mountaineering, a physical tasking repair, or occupations such as firefighter police officer, soldier, or special operator will benefit from increased training efficiency as well as increased safety when facing strenuous activities during training. Signal between about 10 KHz to 1 MHz can be carried sub-dermally throughout the body and aggregated at the central station whether on the patient or remotely monitored. Voltages as low as 5 mV have been shown to produce dermal response, but higher voltages, e.g., about 10 mV, 15 mV, 25 mV, 50 mV, 75 mV, 100 mV, 125 mV, 150 mV, 175 mV, 200 mV, 225 mV, 250 mV, etc., can reduce the effect of external noise sometimes present in the internal dermal transmissions. In addition to these real-time biometric data solutions, the system may incorporate data from other sensors, such as those common on fitness trackers. RF transmission can also be employed. Dermal and/or RF transmission allows the device to be free of hard wire attachments.

Multiple sensor formats are available, each of which embodies a volatile organic compound (VOC) sensing module and associated electronics that access the body's sweat and/or body odor emissions to be used in conjunction with conventional (non-VOC) physiological sensing equipment. All of which is available to the central processor to compile, analyze, record, transmit, compare, and report real-time results of the ongoing activities and to make recommendations for enhanced performance or treatment.

The outputted reports and/or recommendations may be personal to the individual and/or transferred through an advisor, such as a doctor or health care professional, coach, trainer therapist, team captain, etc. (advisor). The reports may be as simple as a sound, LED, or vibration, e.g., to indicate a target level (e.g., level of fat used to support the exercise) is met, intensity should be increased or decreased, goal is met (move on to the next task.), parameter is exceeding set limits, etc. The report may be as complex as desired, e.g., as detailed as a local or remote optical display allows, as text and graph for detailed printed reports, simple up or down arrows, virtual reality headgear, whole body image, etc. When the patient or participant is receiving advice from a supervising professional or an automated program (computer), the advisor or computer may be present with the individual or may be at a remote location. The advisor and/or computer can receive a selected level of programmed or Al enhanced instruction.

When the device in interfaced with an exercise machine, the recommendations may include feedback for the machine, such as if the left or right is stronger, faster, slower, a machine is out of alignment, changed posture, step length, leg speed, exercise session length, frequency or timing, resistance or tension, etc. Recommendations may be provided through an artificial intelligence (Al) engine, that may use individual profile and progress data, average data for the machine or individuals with similar profiles, and/or the individual's historic performance data. For optimal effect, the individual, occupational therapist, physical therapist, trainer, manager, physician or physician's assistant, team captain or other interested party, enters the individual's personal profile that may include goals, number of session, length of program, other equipment available, location—if outdoor activity, pharmaceutical schedule, times of meals, times available for exercise sessions, medical issues or prohibitions. Reports may include text and/or graphics including factors selected from the group consisting of: averages by time of day, interval since last session, effects of meal or meal time, estimate hydration, apparent nutrient or vitamin deficiency, meal suggestions before the next session, etc. The current realtime data are compared with data from previous sessions of that individual and optionally with selected comparative data from similarly profiled (e.g., height, weight, age, gender, temperature, humidity, altitude, exercise program, level of training, injury or body part being addressed, target/goal (e.g., strength, endurance, weight loss), activity or activity mix (e.g., pedaling, running, walking, weights, climbing, specific exercise), ambient temperature, etc. A report is available as desired or permitted in real-time during a training session or may be delivered afterwards to the individual, health care professional, therapist, coach, teammates, etc. The report may be displayed tactilely, audibly and/or visibly. The devices may interface with other reporting, display, or other devices on the individual or on the premises or with remote displays or devices for the individual or the individual's advisor(s).

The present invention provides substantial improvement over existing practices by reporting real-time exercise results for many selectable parameters including, but not limited to: O₂, CO₂, fat metabolism, heart rate, blood pressure, EKG, EEG, chemical energy source, hormones, fatigue, etc., to data obtainable by fitness trackers. Devices of the invention can interface to receive data from many of the available fitness trackers, or comprise such sensing components in a package with the VOC sensing module(s). Grips, bands, apparel, patches, rings, or wraps are featured as easily applied devices, e.g., a grip disposable on a lift bar, step climber, cycle; a headband; a wristband; a belt; a glove; an earplug; a cap or helmet; etc. The VOC sensing module(s) measures and reports current metabolism and thus can see performance and performance improvement or decline. As the metabolism shifts from the glucose in circulation, to induce glycogenolysis and gluconeogenesis, lipolysis, ketogenesis, deamination, VOCs in the circulation shift accordingly. For example, ketones, ketoacids, ammonia, and urea are products from the breakdown of proteins that are accessible using VOC nanosensors in the devices of the present invention. Similarly, activities of insulin and glucagon, are evident in cholesterol pathway metabolism products. These are but two examples of the metabolic signaling observable through VOC sensors. Corticosteroids, their precursors and metabolic products are also observable though monitoring emitted VOCs.

In several embodiments, the present invention features expandable bands that can be slid over or wrapped about a finger, wrist, ankle, foot, head, etc., to collect signal and to communicate with other devices or receivers to inform the exercising individual and/or their teammates or handlers of the effects of the exercise/recovery and the health/fatigue status of the individual. The application of sensing components on a subject is independent of external hardwire connections. The present system offers a thorough and reliable diagnostic understanding of the individual's instantaneous condition with regard to energy utilization, energy source depletion, fatigue, recovery, etc. The status is both immediately and continuously available for providing real-time advice to the athlete and/or therapist, coach, nutritionist, or other interested party—who may interact with other monitors and/or provide feedback instruction to the individual athlete and/or teammates. The system is available with only a simple ability to apply modern technology, e.g., select a device—e.g., a wristband, slip on or strap on the wristband, and select from the desired reported outputs. Optionally, outputs may be transmitted to an associated device for e.g., display on a remote screen, coach or therapist awareness, teammate compensatory action, archiving, etc.

The sensing modules are easily and efficiently applied to or by an individual by simply sliding a band or other securing device (hereinafter: band) over or wrapping it about a location of choice. Alternative embodiments may be presented, e.g., on a piece of exercise equipment, a tool, a baton, etc. The sensors of the present invention avoid any need to apply conductive gel, to penetrate the skin, or to remove hair in order to obtain the desired reporting. The rapid application of these systems allows for its immediate use in training session, a class, a competition, or other situations where real-time metabolic status is beneficial. Because of its low cost, reliability, and effectiveness, the present invention can also be incorporated into standard operating procedures for e.g., monitoring worker fatigue, nutrition status of a person of interest (e.g., infant, person needing assistance, individual with varied work hours, etc.

The invention features a collection of devices of different configurations and purposes from which the individual, instructor, therapist, manager, or coach can select depending on the application. Regardless of format, each bio-informatic device of the present invention provides real-time output for by monitoring human physiology, expressed in the metabolic products. The system is ideally suited for ongoing athletic competition or training. Additional applications for the current invention and its associated systems include, but are not limited to monitoring security workers for alertness/fatigue, truck drivers, crane operators unloading ships, pilots, construction workers, astronauts, etc.

The selection of reporting devices includes bands, tools, grips, handles, batons, pods, etc. In addition to analyzing metabolism expressed in the emitted VOCs, embodiments may also include a “pad” capable of analyzing and reporting results from excreted sweat. Preferred embodiments may also incorporate one or more sensing modules as present in fitness trackers available today.

Several embodiments, feature programmable circuitry to enable auto-selection of both configuration and functionality relevant to its placement location on the body. Preferred embodiments are programmed using artificial intelligence to incorporate identified body location into analysis protocols. Bands or pads may be incorporated within most conventional accessories or clothing including, but not limited to: a helmet, a scarf, a headband, an armband, a wristband, an ankle band, a knee band, chest band, waist band, a pair or shorts, a shirt, a vest, a jacket, etc. Most any securing means are compatible with the present invention. For example, a pad or patch may be using friction, pressure, an adhesive, a hook and loop fastener, elasticity, a clasp, a magnet, a pocket, etc. Exposing an adhesive, for example by peeling off a cover, or simply moving or stretching the device may serve to activate the sensing modules and reporting processes. A membrane, e.g., one permeable to gases in general, or certain sized or chemically excluded gases may isolate a portion of or an entire sensing array. Such membrane may comprise sensors that analyze and output reports relating to results of components that are present in a liquid excretion such as sweat. The membrane, being permeable to gases (including VOCs) permits dual target analysis, the molecules that remain on the liquid side that tend to be larger and the smaller, volatile compounds that can cross the membrane and be analyzed in the gas phase. Multiple arrays of either liquid or gas sensors may be disposed in a single module. Multiple modules may be present in a device. The device may include software to select which of several modules or arrays is position to provide the best data. The device preferably senses when an array or module is damaged and either ignores that array or module in the case that multiple arrays or modules were feeding the report(s) or switches to a second array or module when the first becomes compromised. Devices are designed to communicate through microprocessors and wireless communication with the other devices available to a user. For example, a visual monitor, smart phone, and receiving/transmitting antenna, or earpiece may associate with devices of the present invention to deliver the reports and updates.

Since the preferred analysis target is a human body, sensors require access to the workings of the body in question. To this end, several forms of sensor modules that contact the body to sense and report internal physiologic conditions are necessary. To this end, the present invention includes a selection of devices that contact the skin where liquid excretion (sweat) or gaseous emission (body odor or VOCs) are put in contact with carbon based nano sensors. Such nanosensors have sensing elements whose electronic activity changes when in close proximity (molecular distance scale) to gaseous compounds or compounds carried in the excreted sweat.

Different sensing systems may be mixed and matched to provide desired readouts for chosen factors. Mixtures of sensing systems may be employed to optimize analyses and adaptive responses when programmed for these endeavors. For example, temperature readouts may advise on sustainability of activity in the relevant environment and/or provide adaptations, e.g., warming or cooling to optimize endurance, maximum strength, speed, etc. Devices may include GPS, altimeters, ambient O₂ sensors, mapping (e.g., to find a change or rest point), accelerometers, a photoplethysmography (heart rate and pO₂ that may confirm or adjust the VOC sensing results), gyroscopes, magnetometers, barometers, etc. Data from these auxiliary sensors can be used to help calibrate, confirm or provide additional factors for the device artificial intelligence protocols to use in reporting current status and making suggestions on the level of activity or performance.

Primary embodiments are devices worn by human subjects. A large number of tiny sensors may be incorporated in a device to be worn by the subject of interest. Sensors will typically be formed into arrays, with each array comprising a large number of individual sensor elements. Primary sensors are incorporated into or on one or more surfaces of any wearable device. The sensors are designed and tuned (either electronically or chemically) to be sensitive to one or to a group of gas molecules. Gas molecules of interest include those whose presence or concentration may vary in accordance with the subject's physical status. For example, on a gross scale, ethanol is associated with inebriation; acetaldehyde is associated with a hangover; elevated acetone is associated with diabetes; lactic acid is associated with muscle work, anoxic stress, dehydration, insufficient cardiac output, compromised liver function (e.g., related to stress, fatigue, lack of available fuels), fatigue, etc. Many compounds of analyzable interest belong to the VOC group. As the body supports its activities through metabolic reactions, these reactions will produce organic compounds whose formulations and concentrations are dependent on spontaneous and catalyzed reactions acting on available metabolites. Many of these organic compounds will appear in the circulation and pass into the ambient environment when crossing through skin or evaporating from sweat.

Measuring a single VOC, such as alcohol, may be sufficient to characterize a subject as inebriated. However, generally a reading of several VOCs is more informative.

Many metabolism indicative VOCs have been chemically identified. Identified metabolic VOCs include but are not limited to: acetone, acetophenone, 2-none, 3-buten-2-one, 2-pentanone, 1-phenyl-ethanone, 4-methyl-3-penten-2-one, 3-methyl-cyclopentanone, 6-methyl-5-hepten-2-one, methanol, ethanol, n-propanol, 2-propanol, n-propenal, 2-propenal, 1-butanol, 2-propanol, eucalyptol, 2,4-dimethyl-1-heptene, 2-methyl-pyrazine, 1,3-di-tert-butylbenzene, 1-ethenyl-2,4-dimethylbenzene, 1-methyl-4-prop-1-en-2-ylbenzene, β-pinene, p-cymene, 2,3-dimethylpyrazine, acetaldehyde, benzaldehyde, butyl acetate, butyrolactone, methylmercaptan, isopropyl acetate, isobutyl acetate, cadaverine, methyl tert-butyl ether, ethyl tert-butyl ether, ethylbenzene, carbon monoxide, acetonitrile, dimethyl disulphide, p-menth-1-en-8-ol, dimethyltrisulphide, isoxazolo, α-hydroxybutyric acid, formic acid, acetic acid, propanoic acid, butanoic acid, phenylacetic acid, 8-isoprostane, 2-methyl-1,3-inexpedient, piperidine, limonene, metaxylene, orthoxylene, cyclohexanol, styrene, p-paraxylene, 2-pentylfuran, 2,5-dimethylfuran, 2-methylfuran, 3-hydroxyisovaleric acid, ethyl acetate, isovaleric acid, propane, putrescine, 2,3-dimethyl-2-butene, n-butane, n-butene, 2-butane, 2-butene, pentane, ethene, ethane, 1-propene, 2-propene, n-pentene, 2-methyl-2-pentene, hexane, hexene, heptene, heptane, octane, octene, nonene, nonane, 2-hexanal, heptanal, 2-pentyl nitrate, 3-pentyl nitrate, methyl nitrate, propofol, toluene, n-octanal, p-hydroxyphenylpyruvic acid, n-nonanal, 2-methylpropanal, 2-methylpropenal, butanal, 2-methyl-butanal, 3-methyl-butanal, chloroform, ethylhexanol, 2,3-dihydro-1-phenyl-4 (1H)-quinazolinone, isopropyl myristate, plant or animal pheromones, etc. Alcohols, aldehydes, alkanes, carboxylic acids, fatty acids, and ketones, including substituted versions, are common classes of volatile compounds identified by class but not by specific molecular structure. The artificial intelligence/pattern recognition/machine learning elements (Al) associated with sensing components and applied algorithms may concentrate on one or a plurality of these common metabolites, but will apply the Al to recognize less common or lower concentration metabolites associated with particular disease(s) or conditions. Al will be used to incorporate such findings into the algorithms to achieve improved activities or therapeutic outcomes. “Recognizing” a VOC or VOC pattern is not meant to mean or imply that a chemical structure is known. For example, a profile may in fact recognize a ratio of 3-methyl-butanal to orthoxylene as a factor, but will not confirm the VOC identity or assign molecular structure to the compounds in the ratio. The pattern recognition process identifies interactive patterns of VOC molecules with the sensors. It is the pattern that forms the profile or signature, not any listing of compounds or concentrations—though in some cases the identity of a recognized compound may be determined. The system may also be used for determining levels of stress, breaking points, truthfulness.

The present invention provides capacities for monitoring hundreds or thousands or more of volatile compounds (i.e., compounds that are liberated as gas molecules into the ambient surrounds of the subject). Sensing elements may be protected from water (including heavy perspiration or blood) using selective membranes permeable to gasses but serve as a barrier to penetration by liquid substance. This protective membrane may itself feature its own sensing elements.

The present invention features such sensing elements on a micrometer or nanometer scale. Sensing elements in a range of 10 s of nanometers and larger can be incorporated into clothing or other wearables devices. For example, sizes of individual sensing elements may be in the ballpark of for example, 40 nm, 50 nm, 75 nm, 100 nm, 200 nm, 250 nm, 500 nm, 0.5 μm, 1 μm, 5μm, 10 μm, 50 μm, 100 μm. Shapes may be planar, essentially flat on the substrate surface, or at an angle disposed off the surface. Even if essentially flat, the small size of the sensing elements allows fixation onto curved surfaces, such as a thread in a fabric. Shapes may be irregular, e.g., crumpled or creviced. Shapes may be regular, e.g., hexagonal, circular, triangular, etc.

The sensing elements can be distributed at high density. For example, with a sensor size of 50 nm spaced 50 nm apart, a 1 mm pod of sensors can include 100,000,000 (10⁸). Sensing elements. The extremely high number of available elements allows for redundancies in the case where a sensor is damaged or improperly formed in manufacture.

The sensors communicate with a local and/or with a central processor. Redundancies in communications support longevity, sustainability when damaged, and analytical performances. Local processors may communicate with neighbor local processors and/or processors such as those at a workout facility, maintained by a coach or fitted on one or more teammates.

A plurality of computers can be interfaced to work in or with the system. For example, a NUC computer may process the sensor results in an on board operation. The on body or locally accessible computer may interface hard wired or remotely with one or more adjunct computers, e.g., in a backpack or on a belt, a central processor mounted in a transport device (e.g., a wheeled, airborne (drone monitoring a marathon), flotation device: including, but not limited to scooter, truck, cart, board, capsule, pod, etc.), one or more processors carried on or by a colleague, support animal, robot, etc., a testing station modular set-up, one or more central collection and processing facilities, across outside computational support such as a central command controller or a “cloud” service vendor.

US application, 20170209570, hereby incorporated in its entirety by reference, discloses carbon tube structures including a specific teaching that: “CNTs that form the scaffold may be either single-walled CNTs (SWNTs) or multi-walled CNTs (MWNTs). In a preferred embodiment, the compositions contain SWNTs. SWNTs are formed by a single graphene layer roll-up in the shape of a cylinder. MWNTs are formed by two or more graphene layers rolled-up in the shape of a cylinder. Single-walled carbon nanotubes may assume three types of shapes, termed ‘armchair’, ‘zigzag’, and ‘chiral’, depending on how the six-membered rings are arranged.”

CNTs have been fabricated using several methods including, but not limited to: arc-discharge methods, laser vaporization methods, thermal chemical vapor deposition methods, flowing vapor deposition methods, etc. For example, the arc-discharge method comprises growing CNTs by means of arc discharge from carbon electrodes. This method is capable of producing a tremendous output of CNTs. The laser evaporation method essentially forms CNTs by evaporating molecules off a part of a graphite electrode excited by a laser. The thermal CVD method forms carbon nanotubes at a high temperature by thermally decomposing hydrocarbons used as a carbon source onto a substrate with a predisposed metal catalyst. The flowing vapor deposition method forms carbon nanotubes through an organic transition metal compound and a hydrocarbon compound, transported with a carrier gas, while reacting with one another at a high temperature. Alternative methods of chip manufacture are acceptable for forming sensing elements featured in the present invention.

A preferred sensing device of the present invention is a high sensitivity device that features single walled carbon nanotubules (SWNTs) as a surface to interact with the VOC compounds being evaluated. Other embodiments may feature graphene or synthetic polymers to similar effect. SWNTs and other carbon substrates such as thin or single layer graphene provide both a large surface to volume ratio to facilitate sensor—molecule interaction, and electrical conductivity that facilitate signal transduction. Nano-sensor elements (NSEs}, each including at least one sensing surface, are capable of, for example, of field-effect transistor (FET) or other physico-electrical property/activity. Such structures include, but are not limited to: semi-conducting nano-wires, carbon nano-tubes—including SWNTs, chitosan-cantilever based, synthetic polymers—including dendrimers, plasmon resonance nano-sensors, Förster resonance energy transfer nano-sensors, paramagnetic compounds, surface active crystals, vibrational phonon nano-sensors, magnetically resonant compositions, optical emitting or transforming compositions, optical frequency (or wavelength) based nano-sensors (sensitive to photon transmittance, absorption, reflection, energy modulation, etc.).

SWNTs are especially at differentiating between volatile molecules, generally with molecular weights about 400 g/mol or less. Graphene based substrates have advantages in detecting molecules with an atomic weight of greater than 400 g/mol. may present advantages with respect to liquids or compounds in liquids. The SWNT and graphene-based sensors may be incorporated in a single component package or may work in conjunction with each other. Thus, the platform may, but not necessarily, include sensors that assay larger (non-volatile) molecules.

The single output may connect and thereby collect data signal from any desired fraction of elements. For example, a single output may receive signal from all elements on a chip, half the elements on a chip, one-third the elements on a chip, a quarter the elements on a chip, a fifth the elements on a chip, and so on, for example, ⅙, 1/7, ⅛, 1/9, 1/10, 1/12, 1/20, 1/25, 1/33, 1/50, 1/100, etc. Any output may be associated with 1, 2, 3, 4, 5, 6, 7, 8, 9, 10, 11, 12, 13, 14, 15, 16, 17, 18, 19, 20, 21, 22, 24, 32, 48, 50, 64, 96, 100 (10²), 128, 200, 250, 256, . . . , 500, 512, 1000 (10³), 1024, 2048, 4096, 5000, 8192, 10,000 (10⁴), 16,384, 2¹⁵, 2¹⁶, 10⁵, 2¹⁷, 2¹⁸, 2¹⁹, 10⁶, 2²⁰, total number of sensors on a chip which may vary with time or programmed instructions. The precise count of sensor elements associated with any output in general is a design feature and does not define operative functions of the invention. The counts specifically exemplified above are exemplary low numbers of sensors that may feed an output and higher numbers common in conventional plate assays and powers of 2 and 10 frequently used or approximated in biological or chemical science or physics or electronics. Rectangle configurations such a 3y×4z, where y and z may be independently an integer such as 1 ,2, 3 4, 5, 6, etc., are also common and suitable in arrays of this invention. The VOC sensor elements are preferably nano-sensor elements (NSEs) to minimize size and maximize sensitivity of the sensing chip. NSEs will in general be mounted or carried on a substrate or support matrix forming a “chip”. Individual matrices may feature multiple elements, generally 10 or more, 32 or more, 50 or more, or larger populations of elements on a single chip. As a rule, a greater number on a chip promotes a compactness desired for minimizing weight and size. The number of NSEs is a design feature and can mimic numbers familiar to the operator or data analyst. For example, multiple of the number of wells common on petri dishes may facilitate using existing software tools to further analyze and compare results. Powers of ten, multiples of a hundred or thousand, powers of two are in common use. Accordingly, about 96 elements, 100 elements, 128 elements, 144 elements, 200 or 256 elements, 500 or 512 elements, 10³ or 2¹⁰ elements, 10⁴, 2²⁰, 10⁶, etc., may be built in as common useful working populations even if several elements on the chip are not activated. VOC sensing modules may be independently disposed on one or more patches, pads, etc., or may be integrated into a pad, patch, etc., in conjunction with other sensing devices

NSEs carried on the chips can be any properly designed sensing surface capable of, for example, field-effect transistor (FET) or other physico-electrical property/activity including, but not limited to: semi-conducting nano-wires, carbon nano-tubes—including single-wall carbon nano-tubes, chitosan-cantilever based, synthetic polymers—including dendrimers, plasmon resonance nano-sensors, Förster resonance energy transfer nano-sensors, vibrational phonon nano-sensors, optical emitting, optical frequency (or wavelength) based nano-sensors (sensitive to photon transmittance, absorption, reflection, energy modulation, etc.). Nano FETs and other nano-sensor formats generally operate by changing electrical properties as a substance comes in close proximity to the sensor by perturbing the steady state (absent the proximal substance) charges and movements (distribution of electrons) within the nano-sensor. When the transistor effective electrical properties cause an observable change in electron flow (current) this manifestation is one example of sensor competence. The altered distribution of electrons, depending on the design of the nano-sensor, changes one or more electrical properties, e.g., impedance, resistance-conductivity, capacitance, inductance, etc. and thus the physical movement of a detectable particle, e.g., an electron, a photon, etc.

Minor variances in sensor sensitivities may be weighted internally by the machine learning software or may be overcome by averaging signals of a subpopulation of chips. This massaging feature is available as a tool to promote inter-device and/or inter-chip consistency. The software preferably includes self-calibration and diagnosis for indicating recommended maintenances.

The nanosensing elements can be of a variety of preselected sizes (diameters), heights (due to stacking density), shapes and configurations. Generally, a nanosensing element is decorated with a single species of biomolecule, often a nucleic acid such as RNA or DNA with a length between 8 and 20 nucleotides provides a means for tuning a nanosensor. RNA is another available decoration. The single layered carbon supports generally accept cyclic, especially polycyclic compounds, e.g., porphyrins, phthalocyanines, and azobenzene, as non-covalent associations on their surfaces. Polymers such as the nucleic acids, polyethylene glycol, and fatty acids—especially those conjugated with short polypeptides also form stable surface interactions with the single layered carbon structures and thus are available choices as functional groups on the nanosensing element surfaces. Modified nucleic acids with e.g., nucleotides other than the standard A, C, G, and T and/or incorporation of other molecules in the complex, e.g., stable nucleic acid lipid particles (SNALPS), offer additional choices for detection variations. The assembly of SWNTs decorated with such addons is well-known in the art. Therefore, one skilled in the art does not require a repeated teaching in this paper.

One format extensively described as an example herein involves use of carbon-based structures having properties similar to decorated single wall nanotubules (SWNTs). The carbon component atoms of the nano-tubules are receptive to complexing with ringed chemical structures (decorations or functionalizations), often occurring through a non-covalent π-bonding effect. Graphene, having similar single layer carbon geometry, with proper decoration, can also serve as a sensing surface. Evidence indicates the curved carbon structures of the SWNTs demonstrate more consistent FET properties in many use environments with various functionalization (decoration). Therefore, curved graphene, possibly formed into a corrugated or spiral geometry, (See, e.g., Michael Taeyoung Hwang, et al., Ultrasensitive detection of nucleic acids using deformed graphene channel field effect biosensors. Nat Commun 11,1543 (2020). doi.org/10.1038/s41467-020-15330-9) may demonstrate more promising specificity, speed of analysis, and/or sensitivity over planar graphene for particular applications. As nanotechnology continues to progress additional sensor formats such as those emitting light, will become accepted in the art. Embodiments of the present invention may incorporate these improved sensors as their reliability is established. The skilled artisan will generally choose which form of sensor is optimal for performance and cost.

Nucleic acid is typically applied to nanotubes, or agglomerations thereof, using an aqueous solution which includes buffers and salts under conditions that minimize nucleic acid association or hybridization with other nucleic acids. In such solutions, and in some cases with agitation or sonication, suspensions of agglomerated nanotubes release smaller and smaller agglomerations and eventually produce independent nanotubes with decorative nucleic acid molecules adsorbed on their surfaces.

The binding affinity of biopolymers, with ssDNA as an example, implies that there will be strong binding between the SWNT and the biopolymer. Molecules or volatile compounds bound by the biopolymers on the SWNT will be brought into close contact with the field effect sensor. This affords great compatibility with modern microfabrication techniques, the convenience of electronic readout, small footprint, and ease of fabrication. Useful biopolymers compatible with the present invention include, but are not limited to, polynucleotides such as DNA and RNA, polypeptides, nucleic acid-polypeptide complexes, carbohydrates, aptamers, ribozymes, and all homologs, analogs, conjugates, or derivatives thereof, as well as mixtures thereof. The term “polynucleotide,” generally refers to any polyribonucleotide or polydeoxribonucleotide, which may be unmodified RNA or DNA or modified (non-naturally coding) RNA or DNA. Methylated nucleotides may be used. Polynucleotides include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide encompasses triple-stranded regions comprising RNA or DNA or both RNA and DNA. The term polynucleotide also includes DNAs or RNAs containing one or more modified bases and DNAs or RNAs with backbones modified for stability or for other reasons. Modified bases include, for example, tritylated bases and unusual bases such as inosine. A variety of modifications can be made to DNA and RNA; thus, polynucleotide embraces chemically, enzymatically or metabolically modified forms of polynucleotides as typically found in nature, as well as the chemical forms of DNA and RNA characteristic of viruses and cells. Polynucleotide, as used herein, also embraces relatively short nucleic acid chains, often referred to as oligonucleotides. Incorporating biopolymers onto the nanotubes of the present chemical sensors allows improved sensitivity and selectivity when multiple layers of nanotubes or disposed. This gives the device a gas-sensing functionality that utilizes individualized binding properties of differently decorated nanotubes to differentiate different VOCs. In preferred embodiments of the invention, single stranded DNA is used. These biopolymers provide a comprehensive library of compounds, each with specific binding characteristics.

Selectivity of sensors can be tuned or adjusted by doping or decorating the sensor framing or responsive molecule, e.g., an SWNT whose electronic perturbation affects the circuit electrical activity connecting the associated electrodes. In a typical example, single stranded oligonucleotides robustly pi-bond onto such SWNTs. These are preferred at present because they are easy to produce or obtain commercially and because they offer practically uncountable possibilities for doping. For example, oligonucleotides can be synthesized using nucleotides found in natural genetic materials, but non-natural nucleotide bases may easily be substituted, quickly compounding possibilities available for doping (functionalizing) and differentiating selectivities and sensitivities for assay devices. As an example, US patent application 20100088040, by Johnson, provides a teaching used in the past to create SWNT sensors: “Single stranded oligonucleotides were obtained from Invitrogen® (Carlsbad, Calif.) and diluted in distilled water to make a stock solution of 658 μg/ml (SEQ ID NO: 1) or 728 μg/ml (SEQ ID NO: 2). After odor responses of the bare SWNT-FET device were measured, a 500 μm diameter drop of ssDNA solution was applied to the device for 45 min, and then dried under a nitrogen stream. About 25 devices from two different SWNT growth runs were selected for detailed analysis and treated with ssDNA for the experiments.”

As an alternative to a carbon nanotube base layer, the base layer may be two-dimensional, in the format of a non-tubular graphene. The construction is similar. Rather than constructing carbon nanotubes onto the substrate, graphene, e.g., essentially flat or crumpled is disposed upon the substrate. Then subsequent layers of carbon nanotubes may be applied in a fashion similar to the application atop a ground layer comprising carbon nanotubes.

Graphene detection layers may be used in conjunction with portions or layers of a band that limit evaporation of water, thus providing a liquid for analysis. For example, the thickness of a glove or grip may slow evaporation; a water impermeable, but gas permeable membrane can control water retention.

In addition to the field effect electrical sensing set forth as a preferred embodiment, other qualities of thin carbon based used for sensing are possible. Optical, electrochemical or electrical features have been employed with graphene-based biosensors. Forms of graphene have been successfully tested for electrochemical (amperometric, voltametric, impedimetric, or combinations thereof) and electrical sensing applications. Selected formats have the high electron transfer rate, the high charge-carrier mobility and manageable electrical noise that is necessary for sensitive detection of biomarkers and other biological analytes. Successful assays have been reported in both serum and blood extracts. Optical transparency of graphene monolayers allows use in sensors such as optical-based G-biosensors.

Graphene sensors may be configured as flat, i.e., essentially planar, save for the bend introduced by the chemical bond angles or may be processed to exhibit a thicker, more three-dimensional structure, for example, a folded, rolled, or crumpled graphene. Graphene surfaces may exhibit increased porosity by including gaps or perforations, i.e., discontinuous non-sensor layer portions interspersed within a continuous mesh of structural and/or sensing capable material. Such gaps or perforations may be regularly sized and spaced or may be pseudo-randomly distributed during synthesis. Within a module, layers may incorporate different formats such as synthetic polymer, SWNT, etc. A plurality of liquid and/or gas phase modules is present for improved tunable sensitivity and selectively in some embodiments.

In practice, “essentially flat” graphene structures can be formed as crumpled surfaces or as ultra-flat-surfaces, while tubular graphene, carbon nanotubes (CNTs), or SWNTs exist as tubes. Carbon nanotubes, especially those formed as SWNTs, can be fabricated, and disposed on a substrate to form high surface area scaffolds prepared for the attraction or attachment of interacting molecules such as VOCs. Interacting molecules generally transiently or reversibly interact due to weak attractant forces which transiently disrupt the pattern or cloud of electrons of one or more SWNTs or one or more molecules pre-disposed on (decorating or functionalizing) the sensor element surface. Volatile compounds in close vicinity of a sensor thereby bias electron clouds on the sensor surface and change the electronic effect of the surface sensor molecule. This in turn changes how that molecule electronically interacts with its disposition surface resulting in activation of a Field Effect Transistor (FET) or FET-like effect in associated molecules of the sensor element which service the output electrode(s).

Data from a plurality of devices, preferably from a large plurality of individuals, may be collected and processed to reveal correlative occurrences. For example, data may suggest that one or more combinations of sensed factors, including concentrations, rate of increase, positive and negative correlations, ratios, time delays, activity levels, subject reports, etc., are predictive of or related to beneficial or hazardous outcomes. Such finding obtained in application of artificial intelligence may be instructive in managing subsequent events. A person with similar sensing devices reporting a relevant group of data may receive feedback perhaps to cease, increase, decrease, or modify activity or location to increase performance or to prevent injury to self or others.

Such agglomerated data may be applied in advisory, diagnostic or clinical settings, for example, as indicative of physical or emotional stress, potential for effects of PTSD, agitation, depression, hazardous behavior, amnesia, fatigue, slowed reflexes, allergy, etc. Such associations may be communicated to a subject or a subject's handler(s) so that appropriate action may be communicated to maximize beneficial outcomes.

### DEVICE EXAMPLES

Devices may incorporate sensing elements in accordance with the present invention or be responsive to data contributed by one or more such sensing elements. A wristband, bike handle grip, watchband, ring, bracelet, glove, shoe insert, or similar accessory may collect and process VOC and larger molecule data relating to sweat, VOCs evaporated from sweat and/or VOCs emitted through the skin. Accessory sensors may be incorporated to provide additional data for analysis. For example, temperature of an extremity as well as ambient temperatures may be an important factors for analyzing VOC data. Evaporation of different VOCs will differ by temperature. Ambient temperature may change the body's sweat response. Ratios between different VOCs is expected to vary with temperature as partial pressures of different VOCs may be different, temperature may affect metabolism underneath the skin, and/or temperature may affect delivery of VOCs to the region (e.g., circulation). Other data, for example, blood oxygenation, pulse, etc., may be factors that the algorithms apply during data analysis.

Embodiments can include gloves, grips, bands, etc., that incorporate a heating element. Such heating element may be provided for comfort, e.g., for outdoor workout sessions, but can also serve to increase evaporation (emissions) from the heated body area. For example, bicycle hand grip may be installed in a stationary cycling machine (as common in an occupational or physical therapy clinic). The grips are comfortably warm to the touch and encourage circulation to the palms and fingers. The warmth also enhances VOC emissions for the sensing modules to analyze. The heaters can be powered by any available source of choice. A clinic setup may simply draw power through the programming and display common to such machines; an independent power source such as a battery pack may be selected; the handlebars on a bicycle can house inserts that power the sensors and/or heating elements; a generator, e.g., a wind powered fan or fitting on a front or back wheel; etc. A battery power pack is preferably rechargeable.

In a clinic or other location such as an individual's home, an individual receives their prescription or instruction from a therapist. The individual approaches the equipment, in some circumstances dons a band, fingerlet, or glove, and begins following the prescribed routine. When the equipment is outfitted with the sensor devices and optional accessories, the individual can simply go to the machine and begin training. A therapist present in the clinic, or a therapist at a remote location can monitor the individuals progress on the accessed equipment. Guidance to modify the exercise can be delivered during a session or in prescriptions for subsequent sessions. Guidance may rely solely on the therapists understanding of the individual's performance data (including metabolic performance) or be based on advice from artificial intelligence derived advice relevant to the individual, individuals with similar profiles, the exercise equipment, etc. Most physical/occupational/rehabilitation therapy routines in can benefit from using the present devices. For example, a subject may simply grip a palm sized bar in on or both hands; hand weights can include sensing devices in their grips; equipment using grip bars, e.g., triangle bars, treadmills, resistance machines, etc. Can have their bars outfitted; and subjects can always wear their own or assigned gloves, bands, etc. All exercises, including, but not limited to: cycling, rope pulls, treadmills, lunges, balance ball, grip dynamometer, cuff weights, dumbbells, horizontal and vertical bars, resistance grip, etc., can benefit from analysis of the large and small molecule metabolites and the advisory reports. Many types of clinics or programs can benefit, for example, stroke assessment and recovery, rehabilitation, occupational, and physical therapy; sports training, recovery from micro gravity environment, disability management, etc.

Output from VOC sensing elements is processed to categorize, characterize, quantify and/or identify VOCs. Patterns associated with various stresses, such as dehydration, low blood sugar, muscle fatigue or breakdown, hypo or hyperthermia, oxygen stress, etc., are reported to the individual and/or interested party, such as a coach. Reports may incorporate non-VOC data such as heart rate, blood oxygenation, temperature, breathing rate, perspiration, nearby humans or objects, time spent in activity, distance covered, calories consumed, etc.

Devices of the invention may be secured by any fixture or securing means known in the art, for example, hook and pile like fasteners, tie straps, elastic bands, magnets, adhesives, elastics, etc. As with these devices in general, such equipment mounted devices may be configured for VOC analyses, liquid based analysis and/or any other desired chemical or physical factor.

A coat, a jacket, a partial or full body suit may be configured to incorporate as many of the advantages from more specifically localized accessories as desired.

Fabric or substrate for sensor mounting on a body part may comprise an expandable electro-flexible polymer with sensors spread throughout a large coverage area. Sensors may be disposed in discreet pods or modules interconnected to one another by wired and or wireless circuitry. Individual sensors or pods of sensors may report movement of underlying or associated body parts by being responsive to interactions with neighbor associated sensors or pods.

Inward and outward facing chips report body derived and ambient VOCs, respectively. Ambient temperature and body temperature may be assessed using nano-sensors mounted on a chip. But when desired, ambient temperature may be measured using more conventional methods, e.g., resistance, thermocouple, black body radiation, expansion, temperature sensitive chemicals, expansion interferometry, etc. Photometry, focused inward may report temperature for reading black body radiation, blood oxygenation, skin coloration, pulse oxygenation, etc. An aneroid or other style barometer may report ambient pressure. 3-D or 3-axis accelerometers and/or gyroscopes may be incorporated at one or a plurality of locations to report gravitational and non-gravitational movement and acceleration. Magnetometers, preferably 3-dimensional magnetometers, may be incorporated to report orientations with respect to magnetic fields, e.g., the earth's magnetic polarity.

Temperature, especially temperature difference from ambient temperature or an analogous body part, is indicative of blood flow to the area and may be especially of concern in the extremities where cold may slow circulation and lead to or be indicative of injuries relating to blood flow. Skin coloration is one factor that may be measured with accessory sensors for measuring oxygenation and levels of blood supply to the area monitored.

Each of these devices may incorporate a dedicated microcontroller be connected to a microcontroller connected to other sensing types.

Microcontrollers may operate independently, but preferably communicate with other accessory devices and controllers. A central controller may be independent of the sensing devices but participate in analysis and reporting. Reporting is preferably instantaneous, i.e., real-time. Data from one or more microcontrollers may be stored for later processing or dissemination, e.g., for look-back analysis, or as reference standards for ongoing analyses. Advisory data obtained from devices of the invention may be communicated or displayed to the individual using any conventional means. Light, sound, vibration, squeezing, loosening or tightening a band, electric shock, temperature change, etc., may be instructive to the individual. The signal may be generated by a single device or from processing data from a plurality of devices on that individual or a plurality of individuals. Historical data from the same individual or from a group of similar individuals may be involved in processing the data and reporting outputs.

Algorithms associating stress with VOC profile patterns may be produced using several different protocols, either each independently or in combination. A pattern teaching group can be monitored during daily activities. Changed VOC readings are correlated with stressful situations as they normally occur. Similar data is collected using a confirmation group.

A second protocol involves following persons with stressful activities, e.g., a steel worker, a coal miner, a distance runner, cyclers, skaters, mountaineers, all amateur and professional athletes, any active individual, etc. Specific activity related stresses may be distinct from normal encountered stresses. Accordingly, separate algorithms may be developed to make the reports more relevant to the identified activity and its specific stresses. An a crossover algorithm may recognize both. Stress profiles and/or signatures may be obtained for any stress of interest including, but not limited to physical stress (such as stretch, impact, fatigue, sleep deprivation, nutrient shortage, dehydration, blood loss, head impact, pain, etc.), emotional stress, pain stress, wobbly gait, etc.

### Prototype Examples

A 256 channel NanoArray sensor outputs currents collected by a single chip 256 channel 24 bit analog to digital converter (ADC). This incorporates a very low current signal conditioning with drive electronics for 256 nano-FETs. The chip is controlled by and outputs to a field programmable gate array (FPGA) connected to a high-speed bus connected to e.g., an A10 micro-computer. A wide variety of environment and biophysical sensors are deployable. Each incorporates a specialized chip to convert analog signals directly to digital form for transmission over the high-speed bus.

A foam-like hand grip is placed left and right on a bicycle handlebar. The ‘grips can each operate independently to provide analysis and reporting when only one hand rests on the handlebar. The grips can communicate wirelessly when both are handled to increase the data available for analysis and reporting. In a gym or in a fixed cycling machine, the connection between grips may be hardwired and each cycle may have wired or wireless connection to a central processor that may collate data from all participants. In an example of a team application, e.g., in a bicycle race, each teammate has both handle grips outfitted with a device of the present invention. Each has a receiver accessory that can receive instructions from at least one other unit. In one example format, a captain has a device that receives inputs from all teammates; the captain device may display: a selected relevant factors, a selected series of scrolling factors; and/or multiple factors relevant to a selected team member or team members. The captain, with knowledge of his team members capacities and abilities to push limits, signals the point or front person to allow a new point team member and signals the new point from apparently more rested team members. The system may operate in an automated manner where the system Al analyzes inputs from each member and, e.g., sensing thresholds predicting decreasing performance from a point, signals devices associated with relevant participants for a point change; the fatiguing point maintains a strong effort then drops into the pack when the new point takes over. Embodiments may signal the whole group, just the participants involved in a change, the participants plus a captain, and potentially a support crew. When the system includes historical experience, additional sensors, including, but not limited to: altimeters, anemometers, speedometers, course map, etc., may assist in analyzing the metabolic effort with respect to conditions or expected conditions. Such analysis may be corroborated or performed through comparison to current data from other team members. If a participant is working harder than historical data for the conditions suggests, or harder than peers, the participant and/or support team receive notification to exchange the bike, to favor that teammate, to advise dropping from the pack, to advise leading one last push before dropping back, or to perform maintenance. An optional embodiment includes an enhanced reality visual display showing a full body image of the subject with current status of part shown each body part shown. VOC outputs of body zones may be overlaid on, e.g., local temperature, pO₂, etc. The display might be integral to the device or transmitted to a personal electronic device such as a tablet, smart phone, watch, etc. The device may include an accessory display that could appear as a mirror image, e.g., a full size body image visible to the subject allowing the subject to concentrate on improving specific limbs, muscles, etc.

In place of handle grips, gloves, wristbands, headbands, ankle bands, etc., can serve the same packaging service for the sensor devices. Each sensing device has an analysis component, and preferably a transmitter and receiver. A receiver can be remote from the sensing package, e.g., a screen or earpiece or integrated within. The receiver can be hardwired or wirelessly connected. Receivers may be positioned at select locations on a course, such as hydration station, mile marker, top or bottom of hill, etc. Sensors with short range transmitting capacities, in some embodiments including antenna to provide receive and transmit power, may be placed in ID tags, such as the number tags used to identify participants in a race. The race coordinator can receive alerts when a race participant passes the receiver and transmits data indicating extreme fatigue or a large increase in fatigue when compared to previous checkpoints. Golfers, batsmen, tennis players, etc., with additional sensors to detect speed and change of speed resulting from impact can receive advisories of improved or compromised performance and nutrition suggestions to improve performance. A steering wheel, control stick or other control in contact with the operator can include molecular sensing apparatus. Race drivers, truck drivers, astronauts, etc., (anyone whose occupation may seriously impact safety of themselves or others), can thus be advised to request or to take a rest interval, to refuel, to supplement, etc.

In an exemplary device, oxygen saturation (SpO₂) is measured using a photoplethysmography (PPG) chip (e.g., model ADPD144RI) with red and IR LEDs. Cardiac activity is measured using a pulse pressure detector, or the PPG. This chip also allows respiration measurement. Skin electrical impedance spectra are also measured (up to 200 kHz) are measured. A GPS receiver reports location, speed and altitude. A hygrometer reports humidity. Thermometers report ambient temperature, temperature at the sensor module(s), and/or other location of interest. The graphene-based sensors report larger molecule data. The SWNT sensors report VOC data. A chip stores historical data for that course or route and the individual or similarly profiled individuals. These real-time data are consolidated and compared to the individual's starting data, individual's or course progress data, and historical data from either, to apprise the individual and or other interested parties of health and performance and reserve status. A participant can use the information, e.g., to increase or decrease effort, to seek water, to consume nutrition and/or nutritional supplement. In a cardiology lab, or other highly intensive data gathering environment, the device is applicable to many machine formats, including, but not limited to: treadmill stationary cycle, hand cycle, etc. The cardiology technician and/or cardiologist not only receives the conventional data, e.g., heart rate, blood pressure, pO₂, EKG, but can also monitor metabolism, fuels consumed, cardiac and voluntary muscle derived VOCs, imminent fatigue, etc. During the sensing module derived real-time analysis of the metabolic compounds, an option exists for the associated artificial intelligence engine to incorporate these accessory data in real-time status and final reports. When so applied, these additional data can provide a more complete picture to assess cardiac health and involvement of coronary artery disease. A preferred embodiment includes algorithms that advise the subject, manager, therapist, and/or coach concerning health related background conditions. When the device recognizes VOC patterns associated with a disease or condition or a factor relevant to a condition the subject can be advised to seek appropriate medical counseling. As non-limiting examples, the algorithm(s) might flag cardiac or circulatory stress, e.g., atrial fibrillation, deep vein thrombosis, etc., signatures associated with diabetes, cholesterol levels, etc.

A virtual reality mode can include a mask or headgear, be immersive in an enclosure or cube. Virtual reality solutions may incorporate various accessories, such as sensory gloves, pressure cuffs, full body suit, etc., for simulating real-life or stress situations. Individual response times and compensating activities can be monitored with reference to particular induced stresses. The inclusion of virtual reality also allows practices where individuals facing similar stress inducing situations are trained to respond in manners to modulate the stress response. Observers (human or incorporated Al) overseeing the encounter monitor responses to stresses in real-time to select or control ongoing inputs and analyses during the current or a subsequent session. The subject can in real-time observe responses to stressful situations. With this real-time feedback, the subject can learn accommodative responses. Alternatively, virtual reality can help identify, for a specific individual, expected stress responses and thus to calibrate training management or coaching. A supervisor can use this VR induced information to determine stress situations and understanding maximal stresses specific to an individual. For high stress occupations, monitoring the individual's responses in a virtual reality setting may suggest sub-occupations best avoided. Many variations are conceived for this invention. In general, devices are designed to optimize to augment outcomes relating to one or more therapies or training sessions to result with improving physical performance. The device features a nano-sensing module to detect and monitor volatile organic compounds (VOCs). The module includes a nanosensing element built with a substrate base for an electronic circuit where SWNTs sit between source and drain electrodes disposed upon a dielectric underlayment. The SWNTs carry a current that varies depending on precise intermolecular interactions when a molecule is closely proximate to the sensing surface. Different sensitivities specific to the VOC molecular structures are induced by treating or functionalizing the sensor surface with biomolecules that fix upon the SWNTs and differentially interact with proximal ambient molecules. Nucleic acids are convenient biomolecules for such purpose given their ease of synthesis and the availability of different sequences and lengths. The sensor(s) are served by a microprocessor that collects electronic outputs from the sensor(s) for processing and transmission to an interface that reports the sensed results to one or more devices or human recipients. The VOC status results in a live-time/current health status or may be collated to monitor progression from therapy and/or training. The sensing apparatus is surrounded by a box, band, strap, or other fixture for holding the sensing module and supporting its connections. The device may contain an integral power source, may generate local power, e.g., by mechanical stimulation, and/or may be powered by electromagnetic radiation, a magnetically induced current, etc.

A device may be configured with a plurality of sensing modules. Two or more modules may be activated as determined by an operator. A single module may be adapted or set to a specific function, e.g., fatigue, percent capacity, oxygen, or water stresses, etc. A plurality of modules may be disposed at different locations on the body. For example, separate modules may be set at dispersed zones, maybe the torso, neck, finger, forehead, forearm, wrist, hand, waist, leg, ankle, etc. Each of such multiple device modules may be configured to sense one or more physical status of interest. A module may monitor different situations at different times. Excitement, readiness for activity, may be indicated prior to or at activity onset. Fatigue may be more closely monitored as therapy/training progresses.

Preferred sensor device may be a band, a patch, a ring, a belt, or a grip. Fixtures may be stiff or flexible, such as bendable or elastic. The fixture may be soft and malleable, e.g., sponge like. The sensors may be stand alone, e.g., with wireless communications, may be incorporated into larger devices, such as a cycle grip or other machine handle that may wirelessly or physically connected to the device or a remote consolidator. The grip may be fitted in a stationary bar, e.g. vertically or horizontally secured in a facility, a portable bar such as a walking stick, an exercise bar or stick, etc. The remote consolidation may be in the same room or building or at a distant location. The distant location may, for example be a therapist who provides live advice to the subject or may control the therapeutic machines. Then “therapist” may be a live person, may be an electronic advisor under control an algorithm, preferably being continually updated using artificial intelligence protocols. Remote access may be in a group setting, e.g., remote to other machines that may be located in the same facility as the subject machine, or remote in distant location(s).

1. A device to augment and optimize physical performance, training and therapeutic outcomes, said device comprising:
i) a sensor device comprising a nanosensing module sensitive to volatile organic compounds (VOCs);
said nanosensing module comprising: a nanosensing element comprising a substrate supporting an electronic circuit comprising a) single walled carbon nanotubes (SWNTs) connecting the input and out of said circuit, said SWNTs functionalized with a biomolecule species, b) a microprocessor for receiving, processing and transmitting said data, c) a communicator for accessing said processed data and communicating said processed data to a receiver;
ii) a fixture containing said nanosensing module; and
iii) a connection to at least one power source that when activated energizes said sensing module.

2. The device of claim 1 further comprising:
a second nanosensing module, said second nanosensing module comprising nanosensing element comprising a substrate supporting a second electronic circuit comprising a) an essentially flat graphene layer connecting the input and out of said second circuit, said essentially flat graphene functionalized with a biomolecule species, b) a microprocessor for receiving, processing and transmitting said data, c) a communicator for accessing said processed data and communicating said processed data to a receiver;
ii) a surface containing said nanosensing module; and
iii) a connection to at least one power source that when activated energizes said second sensing module.

3. The device of claim 1 wherein said receiver produces output communication display accessible by an individual in contact with said sensor device.

4. The device of claim 3 wherein said display is selected from the group consisting of: a screen, a vibrating object, a tightening band, a light, a sound, and a temperature change.

5. The device of claim 2 comprising graphene wherein the graphene has a curved or corrugated structure.

6. The device of claim 2 wherein said graphene has a crumpled or irregular structure.

7. The device of claim 1 further comprising a heating element.

8. The device of claim 7 wherein said heating element receives power from harnessing kinetic action inputted by an individual supplying VOCs to said device.

9. The device of claim 1 further comprising a module that provides a virtual reality environment to said individual.

10. A method for improving therapy or training, said method comprising:
z) providing an individual with a device of claim 1;
y) activating said device at an activity station to produce biometric data;
x) processing said biometric data through an artificial intelligence (Al) engine to form a session report; and
w) transmitting said report to a data receiver selected from the group consisting of: said individual, a person controlling, managing, or advising said individual, a device in use or planned for use by said individual, and a database.

11. The method of claim 10, wherein at least one of said device is installed as a hand grip on an exercise machine.

12. The method of claim 11, wherein said exercise machine comprises a bicycle and said at least one hand grip comprises at least one communication port, said at least one communication port capable of communicating with at least one device of at least one teammate or captain.

13. The method of claim 12 wherein said communicating devices apply an artificial intelligence engine to recommend one teammate or captain to front the team.

14. The method of claim 13 wherein said communicating devices accessing changed real-time date recommend a second teammate or captain to front the team.

15. The method of claim 10 further comprising x₂) wherein said Al engine processes data relating to at least one feature selected from the group consisting of: circadian rhythm, time of day, season, time elapsed since latest meal, contents of latest meal, time elapsed since latest awakening from sleep, time elapsed since the initiation of latest menstrual cycle, normal menstrual cycle interval for said individual, pharmaceutical supplements used by said individual, and medical history of said individual.

16. The method of claim 10 further comprising repeating z), and y);
v) accessing in a database said reported biometric data associated with said individual;
u) combining said biometric data from said repeated y) with said reported biometric data associated with said individual;
t) processing said combined data through an Al engine to form a second report; and
s) reporting said second report to a data receiver selected from the group consisting of: said individual, a person controlling, managing, or advising said individual, a device in use or planned for use by said individual, and a database.

17. The method of claim 16 wherein at least two session reports are processed in t) to form said second report said second report.

18. The method of claim 17 wherein said second report processes data combined from session reports in the same time period selected from the group consisting of: a day, a week, a fortnight, a month, a quarter, a preselected number of days, and a year.

19. The method of claim 17 wherein said second report compares session reports from at least a first and a second session.

20. The method of claim 19 wherein said individual suffers an injury between said first session and said second session.
